(19) United States
(12) Patent Application Publication
(10) Pub. No.: US 2022/0313131 A1
(43) Pub. Date: Oct. 6, 2022

(54) DRUG ADMINISTRATION DEVICE

(71) Applicants: Lightnix, Inc., Nishinomiya-shi (JP); Tokyo University of Science Foundation, Tokyo (JP)

(72) Inventors: Noriyuki KOBAYASHI, Nishinomiya-shi (JP); Makiya NISHIKAWA, Tokyo (JP); Kosuke KUSAMORI, Tokyo (JP); Asuka SHIMOMURA, Tokyo (JP); Moeka NISHIMURA, Tokyo (JP)

(21) Appl. No.: 17/642,168

(22) PCT Filed: Sep. 10, 2020
(86) PCT No.: PCT/JP2020/034348

§ 371 Date: Mar. 10, 2022

(30) Foreign Application Priority Data
Sep. 11, 2019 (JP) 2019-165613

(51) Int. Cl. A61B 5/15; A61B 5/151

(52) U.S. Cl. CPC A61B 5/150458; A61B 5/150648; A61B 5/150977; A61B 5/15117

(57) ABSTRACT

A device which, in a shallow region including the epidermal layer or the dermal layer, can administer a target even in extremely small amounts. This includes a puncture needle which, in a flat puncture unit for puncturing the shallow region, has a groove and a through-hole which constitute a recess that extends through part of or the entire thickness of the puncture unit; and a casing which houses the puncture needle so as to allow the puncture needle to advance. By part or all of a flow agent containing the drug being arranged in the groove and the through-hole, the drug is positioned with respect to the puncture unit. The device is designed such that a flow agent in the amount of 10-1000 nL is dosed in the shallow region per puncture.

### TECHNICAL FIELD

The present invention relates to a device which administers a drug to a shallow region including an epidermal layer and/or a dermal layer.

### BACKGROUND ART

Compared to subcutaneous tissues, immune cells such as Langerhans cells and dendritic cells abundantly exist in cutis including dermis, epidermis and the like. It is thus considered that the administration of vaccine to the cutis is more efficient than administration to subcutaneous tissues. A device equipped with a microneedle having a minute needle with a length of less than 1 mm, for example, has been expected to be able to deliver a drug to the cutis.

However, actual examples of intradermal administration of a drug by a device, which can securely puncture the cutis, have not been known. Conventional reports that intradermal administration was performed using an injection needle include cases in which there was a possibility that the needle tip thereof did not stop in the cutis but reached the subcutaneous tissue, and a considerable amount of drug was administered to the skin. Therefore, it is still unclear what amount of drug is administered to the cutis, and how quantitativeness of pharmacological effects of the drug administered to the cutis is.

Meanwhile, Patent Document 1 discloses that by providing a part to be punctured into a living body with a groove or a through-hole, increase in puncture resistance when the needle enters the living body is suppressed, and pain to the living body can be alleviated. Patent Document 1 also describes that, when the needle is punctured with this groove or through-hole being filled with a drug, the drug is easily released.

The device in Patent Document 1, however, is assumed to puncture various places such as cells, tissues, organs, digestive tracks, blood vessels, nerves, skin, muscles and eyes, and when the device punctures particularly the cutis of the skin, e.g. the epidermis and dermis, the amount of drug administered and the quantitativeness of pharmacological effects are unknown.

### Patent Document 1: Japanese Unexamined Patent Application, Publication No. 2015-150216

### DISCLOSURE OF THE INVENTION

### Problems to be Solved by the Invention

The present inventors surprisingly found that when puncturing a shallow region including the epidermal layer and/or the dermal layer with a needle tip to administer a drug to the shallow region, an extremely small amount, sub-microliter order, of drug per puncture was administered to the shallow region, and moreover pharmacological effects were achieved by the extremely small amount of drug administered to the cutis. Based on this finding, there is a potential need for administering an extremely small amount of drug to a shallow region. If an extremely small amount of drug can be administered to the shallow region by one puncture, it can be also expected that the amount of drug administered to the cutis can be finely set by adjusting the number of puncturing.

The present invention was made in view of the above actual circumstances, and an object thereof is to provide a device which can administer a target drug to a shallow region including the epidermal layer and/or the dermal layer, even when an amount of the target drug is extremely small.

### Means for Solving the Problems

(1) A device for administering a drug to a shallow region including an epidermal layer and/or a dermal layer, the device including:

a puncture needle having a recess in a flat-shaped puncture portion to be punctured into the shallow region, the recess extending in a part or entirety of a thickness of the puncture portion, and
a casing for housing the puncture needle so that the puncture needle is capable of advancing,
wherein part or all of a flowable agent including the drug is placed in the recess, so that the drug is positioned with respect to the puncture portion, and
the device is designed to administer the flowable agent in an amount of 10 nL or more and 1000 nL or less per puncture to the shallow region.

(2) The device according to (1), wherein the drug is an antigenic substance.

(3) The device according to (1) or (2), wherein the puncture needle has a means for controlling a depth of puncture into the skin.

(4) The device according to any one of (1) to (3), wherein the puncture needle has two or more puncture portions to puncture the shallow region.

(5) The device according to any one of (1) to (4), wherein the puncture needle has a means for controlling time during which the puncture needle punctures the shallow region.

(6) The device according to any one of (1) to (5), wherein the flowable agent is placed in both the recess and a place other than the recess in the puncture portion.

### Effects of the Invention

According to the embodiments of the present invention, at least part of a flowable agent is placed in a recess provided in a place to be punctured into the shallow region, so that the drug is positioned with respect to the puncture portion. Therefore, when an extremely small amount of flowable agent, 10 to 1000 nL per puncture, is administered to the cutis, a drug included in the flowable agent is easily administered to the cutis with high accuracy.

### BRIEF DESCRIPTION OF THE DRAWINGS

FIG. 1 is a perspective view of a puncture needle according to one embodiment in the present invention;

FIG. 2 is a main part plan view of the puncture needle shown in FIG. 1;

FIG. 3 is an elevation view of the puncture needle shown in FIG. 2;

FIG. 4 are diagrams illustrating positions of a flowable agent placed in the puncture portion of the puncture needle shown in FIG. 1;

FIG. 5 is a perspective view of a puncture needle according to another embodiment in the present invention;

FIG. 6 is a diagram illustrating the position of the flowable agent placed in the puncture portion of the puncture needle shown in FIG. 5;

FIG. 7 is a perspective view of a puncture needle according to one embodiment in the present invention;

FIG. 8 is a cross-section view illustrating an example of a device including the puncture needle shown in FIG. 7;

FIG. 9 is a cross-section view illustrating the action of the device shown in FIG. 8;

FIG. 10 illustrates (A) an optical microscope image of a puncture portion of a puncture needle according to Reference Example, (B) an image obtained by observing a skin sample after puncturing using the puncture portion before embedding, with an optical microscope from the epidermis side, and (C) an image by optical microscopic observation of a section;

FIG. 11 is a diagram illustrating an administration schedule of a flowable agent to mice in the Examples and Comparative Examples; and

FIG. 12 is a graph illustrating changes in mouse immune responses over time in the Examples and Comparative Examples.

### PREFERRED MODE FOR CARRYING OUT THE INVENTION

Embodiments of the present invention will now be described. It should be noted, however, that the present invention is not limited thereto.

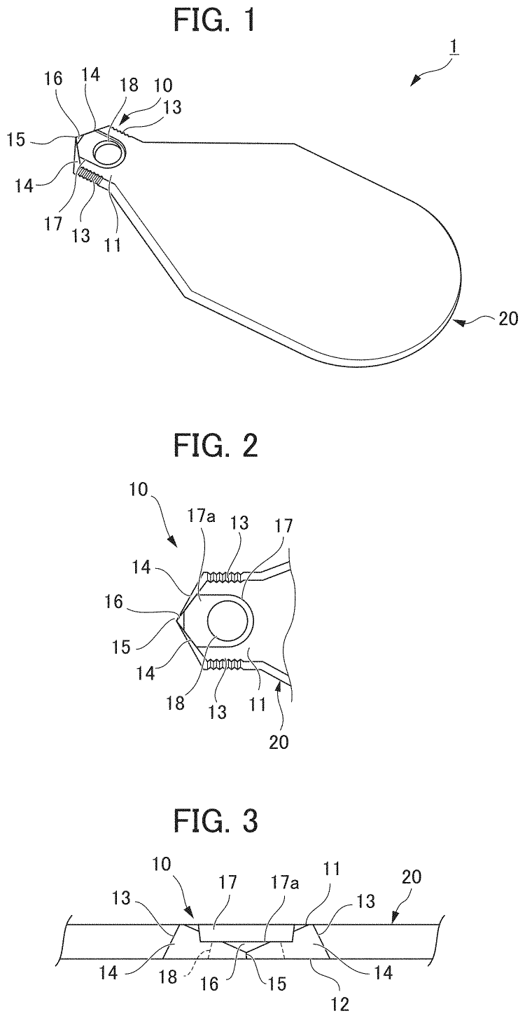

As shown in FIGS. 1 to 3, the puncture portion 10 is formed in a flat shape, the surface and back thereof are a pair of planar parts 11, 12 parallel to each other, and the cross-section perpendicular to the puncture direction is formed in a trapezoidal shape. Examples of the cross-section shape of the puncture portion 10 can include, in addition to the trapezoidal shape, a rectangle shape and multangular shape, and a shape in which the circular or elliptical peripheral surface is cut in the axial direction to form the planar parts parallel to each other, and the like. Serrated concave convex parts 13, 13 are formed in both the sides of the puncture portion 10, by which the penetrating orbit of the puncture portion 10 into the cutis after puncture has high repeatability.

The width of the puncture portion 10 is almost constant in the whole puncture direction in the present embodiment; however, the puncture portion 10 may be formed so that the width will become gradually wider toward the rear. Alternatively, the puncture portion 10 may be formed so that the width will become narrower toward the end (the width will narrow), by which the penetration state of the puncture portion 10 into the cutis is maintained, and the unit is particularly suitable in uses of the present invention in which an extremely small amount of flowable agent, 10 to 1000 nL, is administered to the cutis with high accuracy. Specifically, the width of one puncture portion 10 (maximum width) is preferably 0.5 to 5.0 mm, specifically 1.0 to 2.5 mm.

A pair of inclined side parts 14, 14, which extend at an angle against the puncture direction, are provided on the ends of both the sides of the puncture portion 10 so that the puncture portion 10 will taper. A pair of the inclined side parts 14, 14 are formed in a planar shape almost orthogonal to the surface and back (planar parts 11, 12) of the puncture portion 10. The ends of a pair of the inclined side parts 14, 14 are connected to each other via a linear edge part 15 orthogonal to the surface and back of the puncture portion 10 viewed from the front, and the strength of the edge part 15 is obtained, and puncture in the cutis can be certainly performed.

An inclined surface part 16, which extends at an angle against the puncture direction, is provided on the end of the surface (planar part 11) of the puncture portion 10 so that the puncture portion 10 will taper. The inclined surface part 16 is formed in a planer shape, and the end is connected to the edge part 15. In the present embodiment, the edge part 15 is formed in a liner shape orthogonal to the surface and back of the puncture portion 10 viewed from the front, and the inclined surface part 16 is connected to the one end of the edge part 15; however, the shape of the edge part 15 is not particularly limited. For example, a structure, in which the edge part 15 extends in a line or a curved line along the surface and back of the puncture portion 10 viewed from the front, and the end of the inclined surface part 16 is connected to the whole edge part 15, may be used. Furthermore, a structure, in which the ends of a pair of the inclined side parts 14, 14 and the inclined surface part 16 are connected substantially at one point in the edge part 15, may be used.

The length of the puncture portion 10 is preferably set so that the through-hole 18 can reach a desired depth of the shallow region (normally within about 2 mm from the epidermis) (e.g. about 0.2 to 4 mm, specifically 1 to 2.5 mm). The length required for the puncture portion 10 is dependent on how the puncture portion 10 punctures, and, for example when using elastic expansion and contraction like a coiled spring as described below, the puncture portion 10 moves forward and can start to move backward before the full length thereof penetrates the shallow region depending on the elastic force and the hardness of the cutis. Therefore, the length of the puncture portion 10 can be preferably set to a length larger than the above desired depth.

In the present specification, the shallow region indicates the epidermal layer and/or the dermal layer, and is a region, which is the so-called cutis. Only the epidermal layer may be punctured by the puncture portion 10, or the dermal layer may be punctured through the epidermal layer.

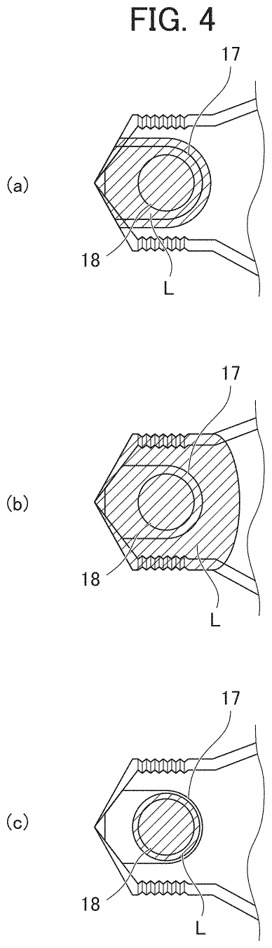

On the surface of the puncture portion 10, a groove part 17, a recess extending in a part of the thickness is formed. The groove part 17 is formed to extend along the puncture direction, and is formed so that the end thereof will cut part of a pair of the inclined side parts 14, 14, and the inclined surface part 16. The back end of the groove part 16 is closed by the side wall. The bottom face 17a of the groove part 17 is formed parallel to the planar parts 11, 12. The side wall of the groove part 17 is formed in a tapered shape so that the width of the groove part 17 will become wider from the bottom face 17a toward the surface (plane surface 11). The groove part 17 is preferably formed to extend over a pair of the inclined side parts 14, 14 and the inclined surface part 16 with the edge part 15 left. In the present embodiment, as shown in FIG. 4, the groove part 17 is formed so that the angle part of the bottom face 17a of the groove part 17 cuts each of a pair of the inclined side parts 14, 14, and the central part of the groove part 17 cuts the inclined surface part 16. A single groove part 17 is preferred; however, a plurality of groove parts 17 can be formed to separately cut a pair of the inclined side parts 14, 14 and the inclined surface part 16.

A through-hole 18 penetrating through the back of the puncture portion 10 (a recess extending in the entirety of the thickness) is formed on the bottom face 17a of the groove part 17. In the present embodiment, a single through-hole 18 is used; however, a plurality of through-holes 18 may be formed on the bottom face 17a of the groove part 17 along or not along the puncture direction. The through-hole 18 may be formed so that the diameter will become wider in a tapered shape from the surface (plane surface 11) toward the back (plane surface 12).

Due to the characteristics of the device, the surface area of the puncture portion 10 is inevitably small, and it is difficult to set a flowable agent containing a drug on a determined place, and additionally, because the flowable agent after setting can move on the puncture portion 10, the repeatability of position of the flowable agent placed is low. When a large amount of flowable agent is administered to a wide range of a living body, the repeatability of position of the flowable agent placed does not easily become a problem; however, when an extremely small amount, 10 to 1000 nL, of flowable agent is administered to a narrow region, the cutis, the repeatability of position of the flowable agent placed can have great influence on the repeatability of administration and therapeutic effects thereof.

Meanwhile, in the embodiments of the present invention, the repeatability of place to set is easily provided by setting the flowable agent based on the groove part 17 and/or the through-hole 18, and also the range of flowable agent moving after setting is physically controlled by the groove part 17 and/or the through-hole 18. That is, when part or all of the flowable agent is placed in the groove part 17 and/or the through-hole 18, the drug included in the flowable agent is positioned in the puncture portion 10. Because of this, an extremely small amount, 10 to 1000 nL, of flowable agent can be administered to the cutis with high accuracy.

At least part of the flowable agent is placed on the surface and/or the back of a place, which is presumed to be punctured to the shallow region, of the puncture portion 10. Because of this, starting from the flowable agent located on the place which has punctured, release is performed from the puncture portion 10 to the cutis, and thus the accuracy and repeatability of administration of the flowable agent to the cutis are increased. To this extent, the flowable agent may be placed in various amounts on various places.

The holding part 20 is formed so that it does not penetrate the cutis, can support the puncture portion 10 outside the skin, and largely expands from the back end of the puncture portion 10. The shape and size of the holding part 20 are not particularly limited.

FIG. 4 are diagrams which show specific aspects of the placement of the flowable agent.

In (a), almost all the flowable agent L is placed in the recess including the groove part 17 and/or the through-hole 18. In (b), part of the flowable agent L is placed in the groove part 17 and the through-hole 18, and the rest of the flowable agent L spreads to the outside of the groove part 17 (to the very end and/or the base of the puncture portion 10). The placement of the flowable agent is not limited thereto, and in an aspect in which the area on which the flowable agent L is exposed to the surface of the puncture portion 10 is larger than the area on which the agent is exposed to the back of the puncture portion 10, when the puncture portion 10 punctures a living body to receive a uniform pressure from the outside, a difference in power which is applied to the flowable agent L from the surface and back of the puncture portion 10 is developed, and the flowable agent filled in the groove part 17 passes through the through-hole 18, and is easily released from the back of the puncture portion 10.

Like (b), the flowable agent L placed in a place other than the groove part 17 and the through-hole 18 is also drawn into the flowable agent L in the groove part 17 and the through-hole 18 by surface tension, and accordingly the position of the flowable agent placed on the puncture portion 10 can be restricted. The flowable agent L placed in a place other than the groove part 17 and the through-hole 18 is released together with the flowable agent L in the groove part 17 and the through-hole 18 from the back to the cutis, and conversely when being directly released into the cutis with which the puncture portion has come in contact at the time of puncture, the flowable agent L can be also released from the surface to the cutis together with the flowable agent L in the groove part 17 and the through-hole 18. That is, the accuracy of administering to the cutis the flowable agent in an amount equal to or higher than the required amount is further increased by placing the flowable agent L on both the recess and a place other than the recess.

On the other hand, as a case where an antigenic substance or an antibody drug is contained, when costs for the flowable agent per unit volume are high, there is a need that the amount of flowable agent placed is reduced as much as possible. For the need, almost all the flowable agent L is placed in the through-hole 18 in (c). The flowable agent set in the through-hole 18 is most restricted to move after setting, and accordingly the repeatability of release to the cutis is highest. Therefore, the aspect of (c) is preferred because the intradermal administration and therapeutic effects according to the amount of flowable agent set can be provided with highest repeatability.

The flowable agent includes a solid (including semisolid), liquid and/or capsule drug, and a flow medium such as liquid, semiliquid, gel and/or sol which dissolve, disperse and/or support the drug. In the case of a flowable agent in which a drug is uniformly dissolved or dispersed in the medium, the distribution of the drug is almost equal to the distribution of the flowable agent; however, in the other cases, the distribution of the drug can be different from the distribution of the flowable agent. For example, it is preferred that a solid (including semisolid) or capsule drug be placed in a certain place of the groove part 17 or the through-hole 18 because the drug is highly positioned in the puncture portion 10. At this time, if there is not a flow medium, it is difficult to regulate whether or not a drug is released to the cutis; however, in the embodiment of the present invention, using a flow medium easily released to the cutis, the medium can be released to the cutis together with the drug with high accuracy. As described above, it is very important that a drug is included in the flowable agent in uses in which an extremely small amount of drug is administered to the cutis with high accuracy.

The drug is not particularly limited and various drugs may be used. Antigenic substances (such as vaccine) are preferred because pharmacological effects can be displayed by administering an extremely small amount thereof to the cutis. In addition, the amount of flowable agent placed is not particularly limited, and may be properly set depending on ease of release to the cutis (which depends on e.g. a place on the puncture portion) and the amount of intradermal administration required, and may be for example 10 nL to 50 mL, particularly less than 10 mL.

In the present embodiment, the through-hole 18 is formed on the bottom face 17a of the groove part 17; however, a structure without the through-hole 18 can be also used. Conversely, a structure in which the through-hole 18 is formed without the groove part 17 can be also used.

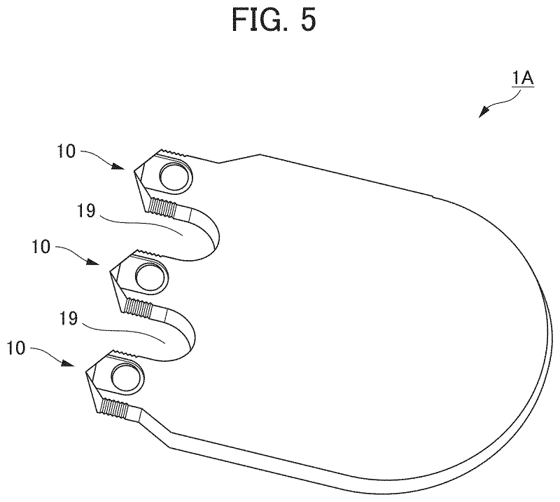

The present embodiment has a structure in which the holding part 20 has a single puncture portion 10; however, as shown in FIG. 5, the holding part 20 of the puncture needle 1A can also have a plurality of puncture portions 10. According to this structure, the plurality of puncture portions 10 can each administer a flowable agent, and thus it is particularly effective when the amount of administration required is high (typically when the amount of flowable agent administered to the cutis per puncture is 100 nL or more, particularly 150 nL or more). The number of puncture portions 10 is not particularly limited; however, the number is preferably 5 or less, 4 or less, 3 or less or two because, when the number is too large, the power transmitted to the edge part of each puncture portion 10 at the time of puncture is dispersed and it is difficult to secure a sufficient puncture depth.

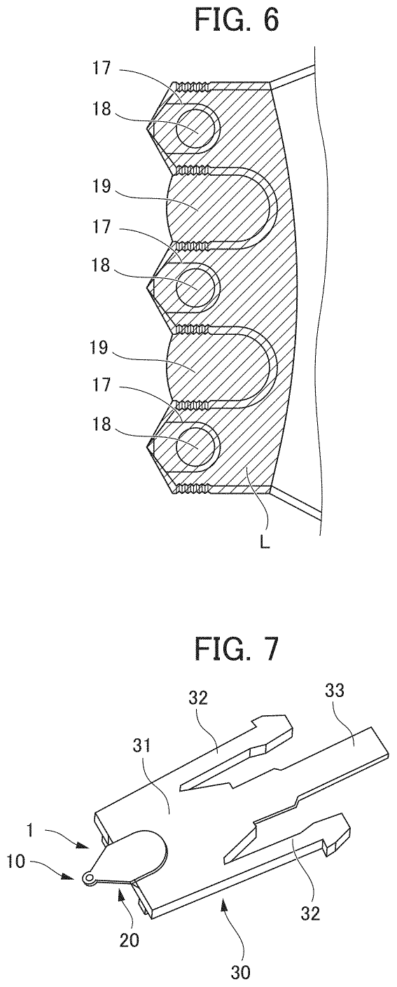

FIG. 6 is a diagram which shows a specific aspect of the placement of the flowable agent L in the puncture needle 1A in FIG. 5. As shown in FIG. 6, the flowable agent L may be also placed in voids 19 between the puncture portions 10. The flowable agent L itself located in the voids 19 is not necessarily administered to the cutis; however, because the flowable agent L placed on each puncture portion 10 makes a large mass together with each other, the repeatability of positions placed is easily realized, and thus this structure is preferred. On the other hand, when it is desired to reduce the amount of flowable agent placed, it is preferred not to place the flowable agent L in the voids 19.

It should be noted that the puncture portions 10 have the same size and shape in FIG. 5; however, they may be different from each other. The drug placed in a puncture portion 10 can be also different from a drug placed in another puncture portion 10. In addition, the flowable agent can be also placed only in some puncture portions 10 of a plurality of the puncture portions 10.

The method for placing the flowable agent L in the puncture portion 10 is not particularly limited, and, for example, a method in which the flowable agent L begins to be added dropwise to a recess (the groove part 17 and/or the through-hole 18) and dropwise addition is stopped at a predetermined volume, or a method in which dropwise addition is stopped immediately before the flowable agent overflows from the recess (the groove part 17 and/or the through-hole 18) or at the time of overflow may be used. Alternatively, the flowable agent L is added dropwise to a place next to a recess (not a recess), and at least part of the flowable agent L may be transferred into a recess (the groove part 17 and/or the through-hole 18) by e.g. tilting the puncture portion 10 from the horizon. In addition, in the case of a solid or capsule drug, the drug is dissolved or dispersed in a medium for the flowable agent L in advance, and then may be set together with the medium in the puncture portion 10, or the drug may be set in the puncture portion 10 separately from the medium before or after setting the medium.

The sizes of the groove part 17 and the through-hole 18 are not particularly restricted; however, each inner volume may be 10 nL or more and about 10 mL. The inner volume of the through-hole 18 depends on the cross-sectional area and depth of the through-hole 18, and the cross-sectional area of the through-hole 18 is restricted to the width of the puncture portion 10, and the depth of the through-hole 18 is restricted to the thickness of the puncture portion 10. A suitable width of the puncture portion 10 is as described above, and the thickness of the puncture portion 10 may be 0.1 mm to 0.9 mm, and is specifically preferably 0.2 to 0.6 mm.

The width of the groove part 17 and the cross-sectional area of the through-hole 18 may be constant in the thickness direction of the puncture portion 10, but they are preferably formed to become wider from the inner part toward the surface or the back in a tapered shape as described in the present embodiment. According to this structure, the contact area between the inner walls of the groove part 17 and the through-hole 18, and the flowable agent can be increased, and the flowable agent can be easily retained in the inside of the groove part 17 and the through-hole 18, and also the flowable agent can be easily administered.

The puncture portion 10 can be produced from a biocompatible material. Examples of the biocompatible material include high molecular polymers, biopolymers, proteins and biocompatible inorganic materials.

As the high molecular polymer, those which can be used for medical cares are preferably used, and examples thereof include polyvinyl chloride, polyethylene glycol, parylene, polyethylene, polypropylene, silicone, polyisoprene, polymethyl methacrylate, fluororesin, polyetherimide, polyethyleneoxide, polyethylene terephthalate, polyethylene succinate, polybutylene terephthalate, polybutylene succinate, polybutylene succinate carbonate, polyphenyleneoxide, polyphenylenesulfide, polyformaldehyde, polyanhydride, polyamide (nylon 6, nylon 6,6), polybutadiene, polyvinyl acetate, polyvinyl alcohol, polyvinylpyrrolidone, polyester amide, polymethyl methacrylate, polyacrylonitrile, polysulfone, polyethersulfone, ABS resin, polycarbonate, polyurethane (polyether urethane, polyester urethane, polyether urethane urea), polyvinylidene chloride, polystyrene, polyacetal, polybutadiene, ethylene vinyl acetate copolymer, ethylene vinyl alcohol copolymer, ethylene propylene copolymer, polyhydroxyethyl methacrylate, polyhydrobutyrate, polyorthoester, polylactic acid, polyglycol, polycaprolactone, polylactic acid copolymer, polyglycolic acid-glycol copolymer, polycapronolactone copolymer, polydioxanone, perfluoroethylene-propylene copolymer, cyanoacrylate copolymer, polybutyl cyanoacrylate, polyallyletherketone, epoxy resin, polyester resin, polyimide, phenol resin and acrylic resin.

Examples of the biopolymer include cellulose, starch, chitin and chitosan, agar, carrageenan, alginic acid, agarose, bullulan, mannan, curdlan, xanthan gum, gellan gum, pectin, xyloglucan, guar gum, lignin, oligosaccharides, hyaluronic acid, sizofiran, lentinan and the like, and examples of the protein include collagen, gelatin, keratin, fibroin, glue, sericin, plant proteins, milk proteins, egg proteins, synthetic proteins, heparin and nucleic acid, and examples include sugar, candy, glucose, maltose, sucrose, mantose, monosaccharides, polysaccharides, and polymer alloys thereof, and the like.

The material of the puncture portion 10 is not particularly limited, and can be selected to obtain desired wettability of the flowable agent, and the wettability can be evaluated by the size of contact angle. The flowable agent can be easily placed in the groove part 17 and the through-hole 18 with high repeatability by desired wettability.

The holding part 20 can be produced using a material the same as or different from the material of the puncture portion 10, and is preferably produced using the same as the material of the puncture portion 10 in an integrated manner. The puncture portion 10 and the holding part 20, for example, can be molded in an integrated manner using mold dies including an upper die and a lower die using a material as described above.

The groove part 17 and the through-hole 18 of the puncture portion 10 can be formed, for example, by injection forming using an upper die and a lower die each including the corresponding projection. Such forming method is particularly effective when the volumes of the groove part 17 and the through-hole 18 are tiny (e.g. 100 nL or less). However, the groove part 17 and the through-hole 18 can be also formed by e.g. laser processing and cutting processing for microfabrication such as excimer laser and femtosecond laser depending on the sizes of the groove part 17 and the through-hole 18.

The puncture needle 1 in the above each embodiment is used as a puncture device which is held in a casing so that the puncture needle 1 can move forward. FIG. 7 is a perspective view which shows an example of a structure in which the puncture needle 1 shown in FIG. 1 is held in a casing and used as a puncture device. The puncture needle 1 shown in FIG. 7 is fixed to the holding body 30 by e.g. integral molding. The holding body 30 includes a main part 31 including the puncture needle 1, a pair of arms 32, 32 provided on both the sides of the main part 31, and a rod 33 extending to the base end of the main part 31.

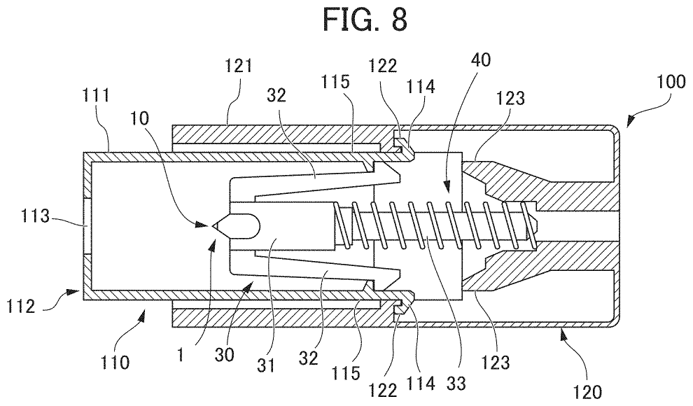

FIG. 8 is a cross-section view which shows an example of a puncture device including the puncture needle 1 shown in FIG. 7. As shown in FIG. 8, the device 100 has a structure in which a holding body 30 including the puncture needle 1 is held in casings including the inner casing 110 and the outer casing 120 so that the holding body 30 can move forward and backward.

The inner casing 110 includes an abutment part 112, which abuts on the surface of a living tissue, on the end of the main part 111, and the inner casing 110 is held in the outer casing 120 so that this abutment part 112 will project to the end side of the outer casing. A projecting opening 113 to project the puncture portion of the puncture needle 1 is formed in the center of the abutment part 112. In addition, the rod 33 of the holding body 30 is inserted into a coiled spring 40.

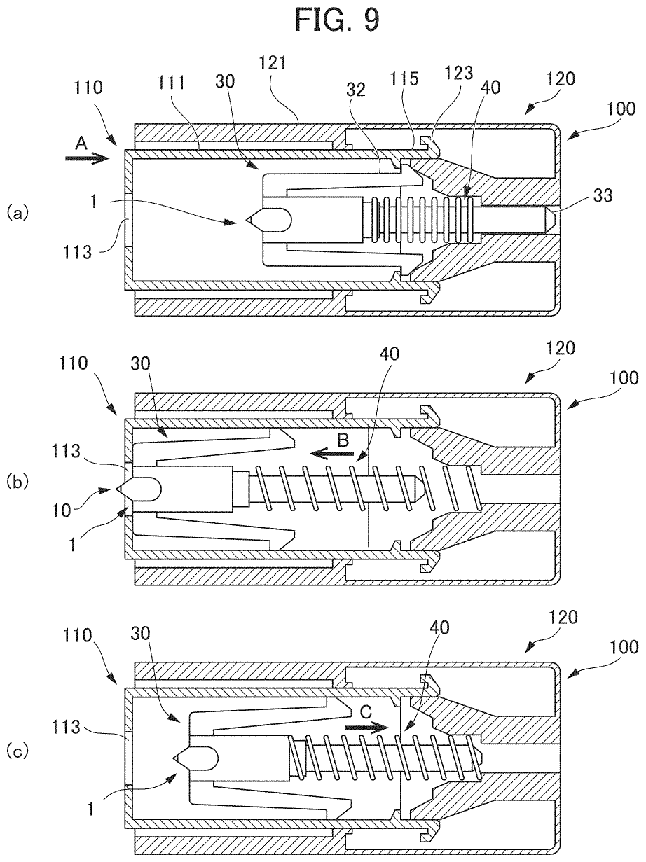

As shown in FIG. 9(a), in the device 100 including the above structure, the inner casing 110 moves backward together with the holding body 30 by squeezing the inner casing 110 into the outer casing 120 in the direction of the arrow A against the spring power of the coiled spring 40, and the spring power of the coiled spring 40 is gradually accumulated. The back ends of a pair of arms 32, 32 in the holding body 30 are curved inward to narrow a gap between them with the accumulation, and the engagement of an engaging part 115 and the arm 32 is released.

When the engagement of the engaging part 115 and arm 32 is released, the holding body 30 vigorously moves to the direction of the arrow B by the spring power accumulated in the coiled spring 40 as shown in FIG. 9(b), and the puncture portion 10 projects through the projecting opening 113 to perform puncture. In this process, when the elastic recoil of the coiled spring 40 is higher than the elastic extension, an increase in the length of projection of the puncture portion is stopped, and when the length starts to return to the natural length, the holding body 30 moves to the direction of the arrow C, and the puncture portion 10 is returned outside the epidermis, and the puncture needle 1 is held in the inside of the inner casing 110 as shown in FIG. 9(c). As described above, the engaging part 115, arm 32 and coiled spring 40 are an example of the method for controlling the depth at which the skin is punctured by the puncture needle, and also an example of the method for controlling the time when the shallow region is punctured by the puncture needle.

By controlling the depth at which the skin is punctured by the puncture needle, the repeatability of a position in the shallow region which is punctured by the puncture portion is increased. When this is coupled with that a drug is positioned on the puncture portion 10, the repeatability of the amount of the flowable agent and the drug administered to the cutis can be dramatically increased. It should be noted that the method for controlling the depth is not limited to the method in which elastic recoil and extension are used in the present embodiment, and a conventionally well-known structure in which when the depth of projection reaches a certain length, the puncture needle is locked may be also used. The depth at which the skin is punctured by the puncture needle can be regulated by the length at which the puncture portion projects from the casing. The length at which the puncture portion projects from the casing may be 0.5 to 2.5 mm, specifically 1.0 to 1.5 mm in unloaded conditions (not at the time of puncture).

By controlling the time when the shallow region is punctured by the puncture needle, the time when the flowable agent on the puncture portion is released to the cutis is controlled, and the repeatability of the amount released can be increased. The time when the shallow region is punctured by the puncture needle can be regulated by controlling the time when the puncture portion projects from the casing. In addition, the time when the puncture portion projects from the casing can be adjusted by e.g. the elastic force of the coiled spring 40. When the time of projection is too short, it is difficult to set the amount of the flowable agent administered to the cutis at a high level, while when the time is too long, it is difficult to set the amount of the flowable agent administered to the cutis at a low level, and thus the time is not particularly limited but may be 0.01 to 0.5 seconds. However, when a structure in which the amount of the flowable agent L released, which is placed on the puncture portion 10, is saturated for a short time (e.g. the amount of the flowable agent L placed is an extremely small amount) is used, even if there is not the above method for controlling the time, the repeatability of the amount released can be sufficient. In addition, the time of puncture may be also controlled by a method other than the device 100 (typically by hand).

However, the device 100 is not limited to the structure in which the puncture needle 1 can move forward or backward in the casing, and a structure in which a medical needle moves forward from the casing and is retained at a puncture position (that is, the medical needle does not move backward after moving forward from the casing) may be also used.

The number of punctures is properly selected from once or twice or more depending on a relationship between the amount of the flowable agent administered per puncture and the total required amount of the flowable agent administered to the cutis. When the number of punctures is twice or more, the number of punctures by one device 100 is basically once, and thus the number of devices 100 which is the same as the number of punctures is normally used; however, one device 100 may puncture several times.

When the number of punctures is twice or more, the places of puncture may be the same or be displaced. However, it was found that when puncture was repeated in a place which had already been punctured or the vicinity thereof, the amount of the flowable agent L released per puncture tended to be reduced. Because of this, it is preferred that the number of punctures be reduced as much as possible (preferably 3 times or less, twice or less or once), or the place of puncture after the second puncture be sufficiently separated from the previous places of puncture.

### EXAMPLES

### Reference Example

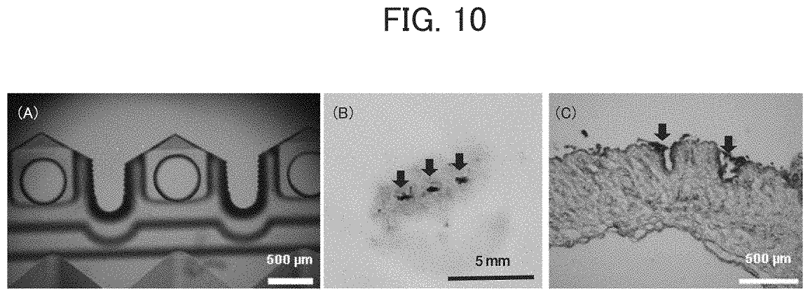

A device was produced by holding the puncture needle shown in FIG. 5 as shown in FIG. 7 and housing it in the casing as shown in FIG. 8. The maximum width of each puncture portion 10 was 4.3 mm, the maximum thickness was 0.45 mm, and the length of the puncture needle projecting from the casing in unloaded conditions was 1.2 mm. The optical microscopic image of the puncture portion is shown in FIG. 10(A).

### (Test)

The back skin of male ddY mice, the hair of which was removed using clippers and a depilatory cream, was excised, and was punctured by the puncture portions of the puncture needle in the device in Reference Example described above. Trypan blue was added dropwise to a region punctured, and after removing excess trypan blue, the skin sample was embedded and frozen in liquid nitrogen. Sections were created from this sample by a cryostat microtome. FIG. 10(B) is an image obtained by observing the skin sample before embedding with an optical microscope from the epidermis, and FIG. 10(C) is an image by optical microscopic observation of the section. The arrows indicate puncture regions.

It was found from FIG. 10(B) that the skin was punctured by all three puncture portions, and from FIG. 10(C) that the depth of puncture was about 250 mm, and the tips of the puncture portions reached the shallow region and did not reach the subcutis.

### Example 1

A device was produced in the same manner as in Reference Example except that 10 mL of flowable agent, an aqueous solution of FITC-dextran (FD4; mean molecular weight 4400), was placed as shown in FIG. 6 by setting the flowable agent from the end of the micropipette to the vicinity of the groove part. In this device, the flowable agent did not move on the puncture portion after setting, and FD4 was positioned in the puncture portion.

### Example 2

A device was produced in the same manner as in Example 1 except that the puncture needle shown in FIG. 1 was used. In this device, the flowable agent did not also move on the puncture portion after setting, and FD4 was positioned in the puncture portion.

### Comparative Examples 1, 2

Devices were produced in the same manner as in Examples 1 and 2 except that the groove part and the through-hole were not provided. In these devices, the flowable agent was set in the same positions as in Examples 1 and 2, but then moved freely on the puncture portion 10, and finally a substantial amount of flowable agent slipped from the puncture portion 10.

### (Test)

The hair on the back of male ddY mice was removed using clippers and a depilatory cream, and the mice were euthanized and then the back skin was excised by a 13 mm punch. The excised skin was punctured by the puncture portion of the puncture needle in the device in Example 1 (once, or three times in total by three devices). The skin surface was lightly wiped, and homogenized in a solution including Triton-X (registered trademark) 100 and NaOH. The amount of fluorescence of FD4 in the supernatant of the centrifuged homogenate was determined by a microplate reader, and based on this, the amount of flowable agent released to the skin was calculated.

Consequently, the amounts released to the skin were 216±37 μL in the site of one puncture, and 351±48 μL in the site of three punctures (n=8, mean value±standard error). Based on the results in FIG. 10 that the puncture portion reached the cutis and did not reach the subcutis, it can be said that the above calculated released amount is the amount administered to the cutis. It was realized and verified for the first time as far as the inventors know that an extremely minute amount, sub-micro order, of flowable agent could be administered to the cutis. It was also found that the amount of flowable agent administered to the cutis could be finely adjusted by selecting the number of punctures by the device in the embodiment of the present invention, and the administration of the flowable agent to the skin was most efficient at the first puncture.

### Example 3

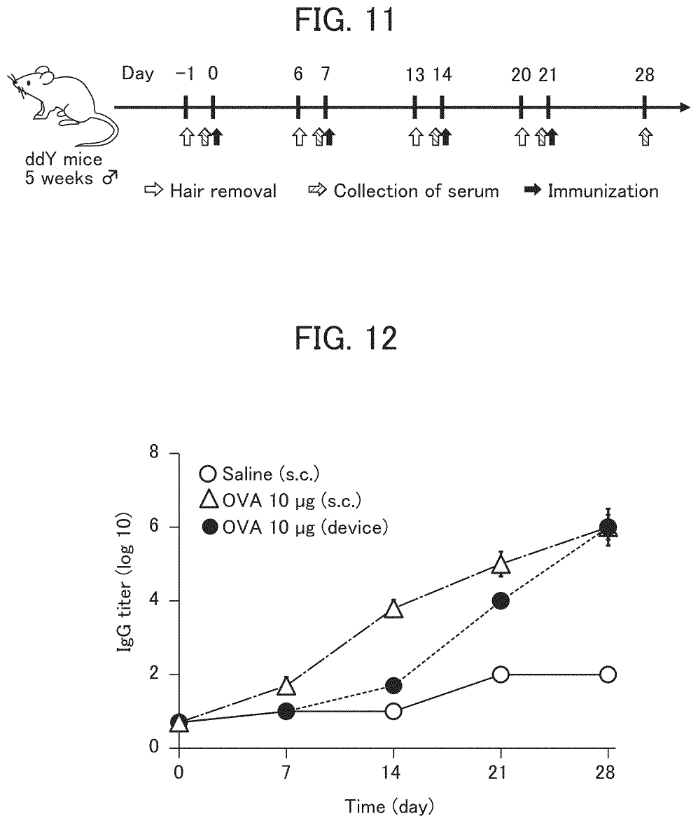

A device was produced in the same manner as in Example 1 except that a saline solution of OVA (1 mg/mL) was used as the flowable agent. Using this device, the flowable agent was administered to the cutis of male ddY mice aged 5 weeks (three punctures in each mouse) by the schedule shown in FIG. 11, and blood serum was collected.

### Comparative Examples 3, 4

Using a commercially available injection needle, 10 mL of the above saline solution of OVA (Comparative Example 3) or the same volume of saline (Comparative Example 4) was subcutaneously injected. The object to be administered and schedule are the same as in Example 3.

The OVA-specific IgG titer of the blood serum collected from mice was measured by ELISA. The results are shown in FIG. 12 (n=4, mean value±standard error). In FIG. 12, closed circle, open triangle and open circle indicate Example 3, Comparative Example 3 and Comparative Example 4, respectively.

From the amount of flowable agent administered to the cutis described in Example 1, it is presumed that a very small amount, about 350 nL, (much smaller than 10 mL in Comparative Example 3) of flowable agent was administered to the cutis also in Example 3. Nonetheless, as shown in FIG. 12, Example 3 and Comparative Example 3 had equal immune responses on the 28th day after starting administration. It was found that as long as even a very small amount, sub-micro order, of flowable agent could be accurately administered to the cutis, sufficient pharmacological effects could be displayed. This fact was discovered for the first time as far as the inventors know.

### EXPLANATION OF REFERENCE NUMERALS

1, 1A: Puncture needle
10: Puncture portion
14: Inclined side part
15: Edge part
16: Inclined surface part
17: Groove part (recess)
18: Through-hole (recess)
20: Holding part
100: Device
110: Inner casing
120: Outer casing

1. A device for administering a drug to a shallow region comprising an epidermal layer and/or a dermal layer, the device comprising:
a puncture needle having a recess in a flat-shaped puncture portion to be punctured into the shallow region, the recess extending in a part or entirety of a thickness of the puncture portion, and
a casing for housing the puncture needle so that the puncture needle is capable of advancing,
wherein a part or all of a flowable agent comprising the drug is placed in the recess, so that the drug is positioned with respect to the puncture portion, and
wherein the device is designed to administer the flowable agent in an amount of 10 nL or more and 1000 nL or less per puncture to the shallow region.

2. The device according to claim 1, wherein the drug is an antigenic substance.

3. The device according to claim 1, wherein the puncture needle has a means for controlling a depth of puncture into skin.

4. The device according to claim 1, wherein the puncture needle has two or more puncture portions to puncture the shallow region.

5. The device according to claim 1, wherein the puncture needle has a means for controlling a time during which the puncture needle punctures the shallow region.

6. The device according to any to claim 1, wherein the flowable agent is placed in both the recess and a place other than the recess in the puncture.
